(19) United States
(12) Patent Application Publication
(10) Pub. No.: US 2010/0137153 A1
(43) Pub. Date: Jun. 3, 2010

(54) Cisplatin-resistance marker for ovarian tumor

(76) Inventor: Moriaki Kusakabe, Ibaraki (JP)

Correspondence Address: ARTHUR G. SCHAIER;CARMODY & TORRANCE LLP, 50 LEAVENWORTH STREET, P.O. BOX 1110, WATERBURY, CT, 06721, US

(21) Appl. No.: 12/290,071

(22) Filed: Oct. 27, 2008

Related U.S. Application Data
(63) Continuation of application No. PCT/JP07/59241, filed on Apr. 27, 2007.

(30) Foreign Application Priority Data
Apr. 28, 2006 (JP) 2006-125086

(51) Int. Cl. C40B 30/04; C07H 21/04; C40B 40/06

(52) U.S. Cl. 506/9; 536/23.1; 536/24.31; 536/24.33; 506/16

(57) ABSTRACT

Disclosed is a cisplatin-resistance marker for determining whether ovarian tumor cells of interest are resistant to cisplatin.

The cisplatin-resistance marker of the present invention includes a polynucleotide having any one of the base sequences of SEQ ID NOs. 1 to 11 and can be used in a detection method involving the following steps (1) to (3) to determine whether ovarian tumor cells of interest are resistant to cisplatin:
(1) hybridizing RNA prepared from a biological sample from a subject, or a complementary polynucleotide transcribed from the RNA, with the cisplatin-resistance marker;
(2) quantifying the RNA from the biological sample, or the complementary polynucleotide transcribed from the RNA, that has hybridized with the cisplatin-resistance marker, using the cisplatin-resistance marker as an index; and
(3) determining whether the biological sample is a cisplatin-resistant ovarian tumor based on the results of the quantification.

### CROSS-REFERENCE TO RELATED APPLICATIONS

This is a continuation of Application No. PCT/JP2007/059241 filed on Apr. 27, 2007.

### BACKGROUND OF THE INVENTION

1. Field of the Invention

The present invention relates to a marker useful in the diagnosis of cisplatin-resistant ovarian tumors and, more particularly, to a marker useful as a probe for the detection of cisplatin-resistant ovarian tumors.

2. Description of the Related Art

Ovarian cancer is a malignant tumor that is one of the major causes of death in women. The disease therefore is a serious concern worldwide. Cisplatin and other platinum-based anticancer drugs are commonly used in the treatment of ovarian cancers. Cisplatin exhibits superior antitumor activity against ovarian cancer.

After prolonged treatment with cisplatin, however, ovarian cancer often loses sensitivity to cisplatin and eventually develops resistance to the drug. Since the cisplatin-resistant ovarian cancer is no longer susceptible to the treatment, additional administration of cisplatin will not produce desired therapeutic effects, but rather will result in side effects. In such a case, the patients may need to consider other therapeutic options. Cisplatin-resistant ovarian cancer and its treatment have been the subject of many studies, as described in Tao Zhang, Ming Guan, Hong Yan Jin, Yuan Lu: Reversal of multidrug resistance by small interfering double-stranded RNAs in ovarian cancer cells. Gynecologic Oncology 2005, 97:501-507; John K. Chan, Huyen Pham, Xue Juan You, Noelle G. Cloven, Robert A. Burger, G. Scott Rose, Kristi Van Nostrand, Murray Kore, Philip J. DiSaia, and Hung Fan: Suppression if ovarian Cancer Cell Tumorigenicity and Evasion of Cisplatin Resistance Using a Truncated Epidermal Growth Factor Receptor in a Rat Model. Cancer Res 2005, 65(8):3242-8; Leigh A. Wilson, Hirotaka Yamamoto, and Gurmit Singh: Role of the transcription factor Ets-1 in cisplatin resistance. Molecular Cancer Therapeutics 2004, 3(7):823-32; and Roohangiz Safaei, Barrett J. Larson, Timothy C. Cheng, Michael A. Gibson, Shinji Otani, Wilturd Naerdemann, Stephen B. Howell: Abnormal lysosomal trafficking and enhanced exosomal export of cisplatin in drug-resistant human ovarian carcinoma cells. Molecular Cancer Therapeutics 2005, 4(10):1595-604.

### BRIEF SUMMARY OF THE INVENTION

Traditionally, whether particular ovarian cancer cells are resistant to cisplatin has been determined by actually exposing the tumor to cisplatin to see if the tumor shows sensitivity to the drug: There has been no way of knowing whether ovarian cancer cells of interest are cisplatin-resistant other than by actually administering cisplatin.

The present inventor compared the gene expression profile of cisplatin-resistant C13 tumor cells with that of cisplatin-sensitive 2008 tumor cells and found that certain genes were specifically expressed in the cisplatin-resistant C13 tumor cells. The inventor also found that the expression levels of some of such genes were increased in the presence of cisplatin and identified these genes (cDNA fragments of SEQ ID NOs. 1 to 11).

The present inventor has found that these genes can serve as marker gene(s) that provide an index of whether ovarian cancer cells (ovarian tumor cells) of interest are cisplatin-resistant. Thus, by using these marker genes, it can be readily determined whether the ovarian cancer cells are resistant to cisplatin. The present invention was accomplished based on these findings.

According to the present invention, there is provided a cisplatin-resistance marker for ovarian tumor that includes a polynucleotide having any one of the base sequences of SEQ ID NOs.1 to 11.

There is also provided a cisplatin-resistance marker that includes a polynucleotide having a base sequence that hybridizes with the above-described polynucleotide under stringent conditions.

Further, there is also provided a cisplatin-resistance marker that includes a polynucleotide having a base sequence complementary to all or part of the above-described polynucleotide.

Each of the above-described cisplatin-resistance markers may be used as a probe to detect a cisplatin-resistant ovarian tumor.

Each of the above-described cisplatin-resistance markers may be used as a primer to detect a cisplatin-resistant ovarian tumor.

According to the present invention, there is also provided a microarray for detecting a cisplatin-resistant ovarian tumor that includes any of the above-described cisplatin-resistance markers.

According to the present invention, there is also provided a method for detecting a cisplatin-resistant ovarian tumor. The method includes (1) hybridizing RNA prepared from a biological sample from a subject, or a complementary polynucleotide transcribed from the RNA, with the above-described cisplatin-resistance marker; (2) quantifying the RNA from the biological sample, or the complementary polynucleotide transcribed from the RNA, that has hybridized with the cisplatin-resistance marker, using the cisplatin-resistance marker as an index; and (3) determining whether the biological sample is a cisplatin-resistant ovarian tumor based on the results of the quantification.

The present invention makes it possible to determine, without administering cisplatin, whether a given ovarian cancer is resistant to cisplatin.

### BRIEF DESCRIPTION OF THE SEVERAL VIEWS OF THE DRAWINGS

FIG. 1 is a flowchart showing the procedure of HiCEP technique.

FIG. 2 is a graph showing the expression levels of the gene of SEQ ID NO: 1, as measured by real-time PCR.

FIG. 3 is a graph showing the expression levels of the gene of SEQ ID NO: 2, as measured by real-time PCR.

FIG. 4 is a graph showing the expression levels of the gene of SEQ ID NO: 3, as measured by real-time PCR.

FIG. 5 is a graph showing the expression levels of the gene of SEQ ID NO: 4, as measured by real-time PCR.

FIG. 6 is a graph showing the expression levels of the gene of SEQ ID NO: 5, as measured by real-time PCR.

FIG. 7 is a graph showing the expression levels of the gene of SEQ ID NO: 6, as measured by real-time PCR.

FIG. 8 is a graph showing the expression levels of the gene of SEQ ID NO: 7, as measured by real-time PCR.

FIG. 9 is a graph showing the expression levels of the gene of SEQ ID NO: 8, as measured by real-time PCR.

FIG. 10 is a graph showing the expression levels of the gene of SEQ ID NO: 9, as measured by real-time PCR.

FIG. 11 is a graph showing the expression levels of the gene of SEQ ID NO: 10, as measured by real-time PCR.

FIG. 12 is a graph showing the expression levels of the gene of SEQ ID NO: 11, as measured by real-time PCR.

### DETAILED DESCRIPTION OF THE INVENTION

Unless otherwise specified, the term “gene (DNA)” as used herein is intended to encompass not only double-stranded DNA, but also respective single stranded DNA molecules known as sense and anti-sense strands that together form a double stranded DNA molecule. The term “gene (DNA)” may refer to either a structural gene or a regulatory gene. Aside from human gene (DNA), the term “gene (DNA)” also encompasses genes of non-human origin, such as mice and rats (homologues), that do not interfere with the objective of the present invention.

Unless otherwise specified, the term “polynucleotide” as used herein is intended to encompass both DNA and RNA. Unless specified, the term “DNA” is intended to encompass cDNA, genomic DNA and synthetic DNA. Unless specified, the term “RNA” is intended to encompass any of total RNA, mRNA, rRNA and synthetic RNA.

### (Polynucleotides)

The Polynucleotides Shown by SEQ ID NOs. 1 to 11 are Obtained from cisplatin-resistant C13 tumor cells and are specifically expressed in cisplatin-resistant C13 tumor cells.

Specifically, the cisplatin-resistant C13 tumor cells were obtained from a solid tumor formed in vivo by inoculating a cisplatin-resistant ovarian cancer cell line (C13•5.25/2008, cisplatin-resistant C13 cell line) into the dorsal skin of nude mice. The cisplatin-resistant C13 tumor cell line was established by culturing a cisplatin-sensitive ovarian cancer cell line (2008 cell line) for 13 months with increasing concentrations of cisplatin according to the technique described by Andrews P A et al. (Andrews P A, et al: Differential potentiation of alkylating and platinating agent cytotoxicity in human ovarian carcinoma cells by glutathione depletion. Cancer Res 15; 6250-6253, 1985). The cisplatin-sensitive ovarian cancer cell line is a cell line established by DiSaida P. J. from serous cystadenocarcinoma of the ovary (DiSaida P J, et al.: Cell-mediated immunity to human malignant cells. Am J Obstet Gynecol 114; 979-989, 1972.).

By saying that a gene is expressed “specifically” in the cells of the above-described tumor formed by inoculating the cisplatin-resistant ovarian cancer cell line into the dorsal skin of nude mice C13 tumor), it is meant that the gene meets all of the following criteria:
its peak as measured by high-coverage expression profiling (HiCEP) is 3 times or higher for the C13 tumor than for the tumor formed by inoculating the cisplatin-sensitive ovarian cancer cell line into the dorsal skin of nude mice (2008 tumor);
its expression level as measured by real-time PCR is 2 to 5 times or more higher in the C13 tumor than in the 2008 tumor; and
its expression level further increases in the presence of cisplatin.

The polynucleotides shown by SEQ ID NOs.1 to 11 are obtained by subjecting the polynucleotides in the cisplatin-resistant C13 tumor cells to the primary screening and secondary screening as described below. These sequences have been deposited in GenBank under the following accession numbers: AC091010 (SEQ ID NO: 1), NM_{—}003739 (SEQ ID NO: 2), ACO₂₆₇₂₂ (SEQ ID NO: 3), NM_{—}205845 (SEQ ID NO: 4), AC079136 (SEQ ID NO: 5), BF059583 (SEQ ID NO: 6), BX470629 (SEQ ID NO: 7), AY613922 (SEQ ID NO: 8), NM_{—}001873 (SEQ ID NO: 9), AL034419 (SEQ ID NO: 10) and NM_{—}001266 (SEQ ID NO: 11).

### (Primary Screening)

The genes specifically expressed in the cisplatin-resistant C13 tumor cells were identified by HiCEP (high-coverage expression profiling) technique and sequenced. HiCEP is a technique developed by Fukumura R. et al. (Fukumura R. et al.: A sensitive transcriptome analysis method that can detect unknown transcripts. Nucleic Acids Res. 2003 Aug. 15; 31(16):e94. Reference may also be made to International Publication No. WO 02/048352 pamphlet, Japanese Patent Application Laid-Open (JP-A) No. 2005-006554, and Japanese Patent Application Laid-Open (JP-A) No. 2005-250615).

Using HiCEP, mRNA (cDNA) expressed in the cisplatin-resistant C13 tumor cells and mRNA (cDNA) expressed in the cisplatin-sensitive 2008 tumor cells were analyzed and compared with each other to identify genes specifically expressed in the cisplatin-resistant C13 tumor cells.

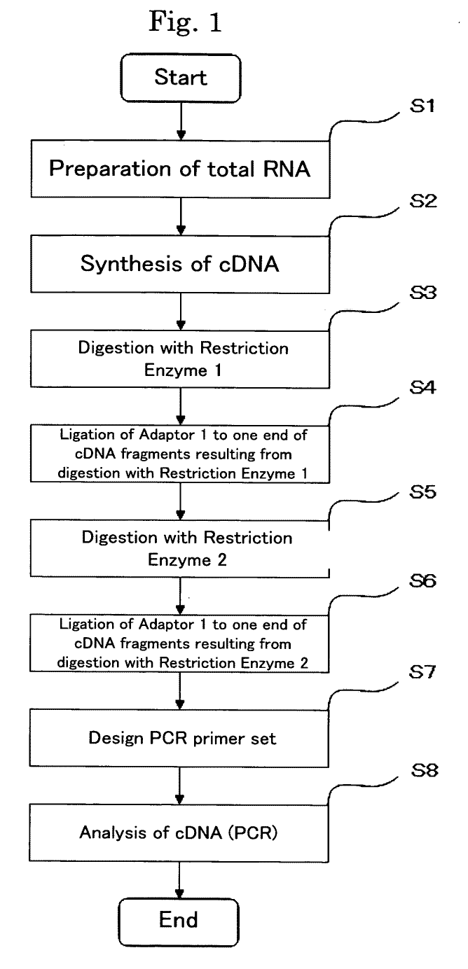

As shown in FIG. 1, the HiCEP procedure generally includes 8 steps. First, total RNA was extracted from both the cisplatin-resistant C13 tumor cells and the cisplatin-sensitive 2008 tumor cells (C13 total RNA and 2008 total RNA) in Step 1 (S1).

Total RNA of tumor cells can be prepared by known techniques. To prepare total RNA in the present embodiment, tumors were first created in nude mice by inoculating the 2008 cells and C13 cells. The 2008 and C13 tumors were excised and lysed to form a lysate. Specifically, the 2008 tumors were created by subcutaneously inoculating the cultured 2008 cells into the dorsal skin of nude mice at 3×10⁶ cells/mouse and allowing them to grow for 3 weeks. Likewise, the C13 tumors were created by subcutaneously inoculating the cultured C13 cells into the dorsal skin of nude mice at 5×10⁶ cells/mouse and allowing them to grow for 8 weeks. Using a special kit (RNeasy Mini Kit, Quiagen, cat. No. 74104), total RNA was prepared from the resulting lysates. QIAshredder (Quiagen, cat. No. 79654) was used to completely homogenize the lysates and RNase-Free DNase Set (Quiagen, cat. No. 79245) was used to remove the genomic DNA contaminants. To prepare the total RNA, the kit was used according to the accompanying instruction manual.

In Step 2 (S2), cDNA (double-stranded) is synthesized from the total RNA from Step 1. First, mRNA in the total RNA was reverse-transcribed into cDNA (single-stranded cDNA) using a biotin-labeled poly dT oligomer as a primer. After the synthesis of single-stranded cDNA, DNA polymerase, E. coli ligase and RNaseH were used in a standard technique to synthesize the complementary strand, thus forming double-stranded cDNA.

In Step 3 (S3 in FIG. 1), cDNA obtained in Step 2 is digested with a first restriction enzyme (Restriction Enzyme 1). Restriction Enzyme 1 used in the present embodiment was MspI (recognition sequence: CCGG).

In Step 4 (S4 in FIG. 1), a first adaptor (Adaptor 1) is ligated to the cDNA fragments resulting from digestion with Restriction Enzyme 1. Adaptor 1 used in the present embodiment was MspI-adaptor.

MspI-adaptor has the following base sequences:

5′-aatggctacacgaactcggttcatgaca-3′ (SEQ ID NO: 12) and

5′-cgtgtcatgaaccgagttcgtgtagccatt-3′ (SEQ ID NO: 13).

Using T4 ligase, MspI-adaptor was ligated to the cDNA fragments resulting from digestion with MspI. Only the biotin-labeled cDNA fragments were then collected by binding to streptavidin magnetic beads.

In Step 5 (S5 in FIG. 1), the cDNA fragments obtained in Step 4 are digested with a second restriction enzyme (Restriction Enzyme 2). Restriction Enzyme 2 used in the present embodiment was MseI (recognition sequence: TTAA). In Step 5, the cDNA fragments bound to streptavidin magnetic beads were digested with MseI. The free cDNA fragments released from the magnetic beads after digestion with MseI were collected.

In Step 6 (S6 in FIG. 1), a second adaptor (Adaptor 2) is ligated to the cDNA fragments resulting from digestion with Restriction Enzyme 2. Adaptor 2 used in the present embodiment was MseI-adaptor.

MseI-adaptor has the following base sequences:

5′-aagtatcgtcacgaggcgtcctactgcg-3′ (SEQ ID NO: 14) and 5′-tacgcagtaggacgcctcgtgacgatactt-3′ (SEQ ID NO: 15).

Using T4 ligase, MseI-adaptor was ligated to the cDNA fragments resulting from digestion with MseI. The resulting cDNA fragments had the MspI adaptor ligated to one end and the MseI adaptor to the other end thereof.

In Step 7 (S7 in FIG. 1), a PCR primer set is designed to amplify the cDNA fragments obtained in Step 6 having the MspI-adaptor ligated to one end and the MseI-adaptor to the other end thereof. In the present embodiment, a primer having a base sequence complementary to the MspI adaptor (first primer) and a primer having a base sequence complementary to the MseI adaptor (second primer) were prepared using a known technique. Only the first primer was fluorescence-labeled (for example, with 6-carboxyfluorescein, FAM), so that the cDNA by-products having MseI-adaptor ligated to each end that may be present in the cDNA fragments resulting from Step 6 will not be detected after the PCR.

The first and second primers used were MspI primer and MseI primer with the following base sequences, respectively:

MspI primer: 5′-label-actcggttcatgacacggnn-3′ (SEQ ID NO: 16)

MseI primer: 5′-aggcgtcctactgcgtaann-3′ (SEQ ID NO: 17).

In Step 8 (S8 in FIG. 1), PCR is performed using the cDNA fragments obtained in Step 6 and the primer set obtained in Step 7. In this step, cDNA fragments (or mRNA) expressed at high levels can be analyzed.

Based on the results obtained by HiCEP, the cDNA (mRNA) expression profile was compared between the cisplatin-sensitive 2008 tumor cells and the cisplatin-resistant C13 tumor cells to identify 71 different cDNA fragments that were specifically expressed in the cisplatin-resistant C13 tumor cells.

The resulting 71 cDNA fragments were sequenced in the following manner: Samples identical to those used in the HiCEP procedure above were electrophoresed on slabs of sequencing acrylamide gels (gel concentration=4% for bands for 300 bp or larger molecules, 6% for bands for molecules sized 130 bp to 300 bp, and 10% for bands for 130 bp or smaller molecules). The gels were read by a fluorescence scanner and the desired bands for HiCEP were excised by superimposing the radiograph with each gel. Using the same primers as those used in HiCEP, the excised DNA bands were amplified and the amplified 71 cDNA fragments were sequenced by direct sequencing (ABI3100, Applied Biosystems).

### (Secondary Screening)

In the secondary screening, the 71 genes identified in the primary screening were screened for their expression levels in the presence of cisplatin. Specifically, of the 71 genes, those that were expressed at higher levels in the presence of cisplatin than in the absence of cisplatin were selected. This is based on the speculation that the genes whose expression levels increase in the presence of cisplatin are truly specifically expressed in the cisplatin-resistant C13 tumor cells. The procedure of the secondary screening are as described below.

Total RNA was extracted from the cisplatin-resistant C13 tumor cells grown with or without exposure to cisplatin. Specifically, the cisplatin-resistant C13 tumor cells without cisplatin exposure (referred to as C13 (−), hereinafter) were obtained from the tumor created by subcutaneously inoculating cisplatin-resistant C13 cells (5×10⁶ cells/mouse) in the dorsal skin of nude mice (mice were intraperitoneally administered physiological saline after a 8-week growth period). The cisplatin-resistant C13 tumor cells with cisplatin exposure (referred to as C13 (+), hereinafter) were obtained by subcutaneously inoculating cisplatin-resistant C13 cells in the dorsal skin of nude mice to form a tumor, and intraperitoneally administering cisplatin at a dose of 15 μg/1 g body weight after a 8-week growth period. Total RNA was extracted from the C13 (−) and the C13 (+) in essentially the same manner as the above-described extraction of total RNA. For comparison, total RNA was also extracted from the cisplatin-sensitive 2008 tumor cells grown with or without exposure to cisplatin. Specifically, the cisplatin-sensitive 2008 tumor cells without cisplatin exposure (referred to as 2008 (−), hereinafter) were obtained from the tumor created by subcutaneously inoculating the cisplatin-sensitive 2008 cells (3×10⁶ cells/mouse) in the dorsal skin of nude mice (mice were intraperitoneally administered physiological saline after a 3-week growth period). The cisplatin-sensitive 2008 tumor cells with cisplatin exposure (referred to as 2008 (+), hereinafter) were obtained by subcutaneously inoculating the cisplatin-sensitive 2008 cells and intraperitoneally administering cisplatin at a dose of 15 μg/1 g body weight after a 3-week growth period.

Using a commercially available special kit (SuperScriptIII First-Strand Synthesis for RT-PCR, Invitrogen, Cat. No. 18080-51), cDNA solutions were prepared from the total RNA samples of C13 (−), C13 (+), 2008 (−) and 2008 (+). The solutions were prepared as described below.

For each total RNA sample, 5 μg of total RNA, 1 μl of 50 μM oligo(dT)20, and 1 μl of 10 mM dNTP mix were placed in a tube and DEPC-treated water was added to a total volume of 10 μl. The reaction was incubated at 65° C. for 5 min and then rapidly cooled on ice (1 min). Subsequently, a mixture having the following composition was added:

(Composition of mixture)

| | |
|---|---|
| 10 x RT buffer | 2 μl |
| 25 mM MgCl₂ | 4 μl |
| 0.1M DTT | 2 μl |
| RNaseOUT (40 U/μl) | 1 μl |
| SuperScriptIII RT (200 U/μl) | 1 μl |

After addition of the mixture, the reaction was incubated at 50° C. for 60 min, then at 85° C. for 5 min. RNase H (1 μl) was added and the reaction was incubated at 37° C. for 20 min to obtain a desired cDNA solution (stored at −20° C.).

The 4 different cDNA solutions obtained above were analyzed by real-time PCR (7500 Fast Real Time PCR System; 7700 Fast Real Time PCR System, Applied Biosystems (ABI)). Given below is the composition of the mixture analyzed.

(Composition of real-time PCR mixture)

| | |
|---|---|
| SYBR Green PCR Master Mix (Cat. No. 4309155, ABI) | 10 μl |
| Forward primer (10 pmol/μl) | 1.8 μl |
| Reverse primer (10 pmol/μl) | 1.8 μl |
| Distilled water | 4.4 μl |
| cDNA solution | 1 μl |

The forward primers (FP) and reverse primers (RP) used in the analysis had the following sequences.

### [For SEQ ID NO: 1]

| | | |
|---|---|---|
| FP (s2-2f): | cggattggagtgtcttaacg | (SEQ ID NO: 18) |
| RP (s2-2r): | cagccaccatagcaggaaca | (SEQ ID NO: 19) |

### [For SEQ ID NO: 2]

| | | |
|---|---|---|
| FP (s8-2f): | ttccagttgactgcagagga | (SEQ ID NO: 20) |
| RP (s8-1r): | tcgctaaacaggacggattt | (SEQ ID NO: 21) |

### [For SEQ ID NO: 3]

| | | |
|---|---|---|
| FP (s19-2f): | tgtagatggcaggttgatgg | (SEQ ID No: 22) |
| RP (s19-2r): | ggttaggggtctgatgagca | (SEQ ID NO: 23) |

### [For SEQ ID NO: 4]

| | | |
|---|---|---|
| FP (b7-1f): | cttactgaagtcgccaagca | (SEQ ID NO: 24) |
| RP (b7-1r): | tgtgcgatatttgacccttg | (SEQ ID NO: 25) |

### [For SEQ ID NO: 5]

| | | |
|---|---|---|
| FP (s22-2f): | cggagctgcaatctagtcct | (SEQ ID No: 26) |
| RP (s22-2r): | attcgccacagcttttcaat | (SEQ ID NO: 27) |

### [For SEQ ID NO: 6]

| | | |
|---|---|---|
| FP (s24n-2f): | atgctgctgtgaaagtgtgc | (SEQ ID No: 28) |
| RP (s24n-2r): | caggctgggtttcttctctg | (SEQ ID NO: 29) |

### [For SEQ ID NO: 7]

| | | |
|---|---|---|
| FP (s43-1f): | ggcaggaatgaaacaggaaa | (SEQ ID No: 30) |
| RP (s43-1r): | gatttcgttgaccccatcac | (SEQ ID No: 31) |

### [For SEQ ID NO: 8]

| | | |
|---|---|---|
| FP (s47a-1f): | ccgacctgaaaccatctctg | (SEQ ID NO: 32) |
| RP (s47a-1r): | aagggctttctctcaatcct | (SEQ ID NO: 33) |

### [For SEQ ID NO: 9]

| | | |
|---|---|---|
| FP (s65-5f): | gctcctggtcatcgagctgt | (SEQ ID NO: 34) |
| RP (s65-5r): | ttgtctcgttccccttctgg | (SEQ ID NO: 35) |

### [For SEQ ID NO: 10]

| | | |
|---|---|---|
| FP (b49-2f): | tggagaagaaggtccctcaa | (SEQ ID NO: 36) |
| RP (b49-1r): | gggcagggattagagtctcc | (SEQ ID NO: 37) |

### [For SEQ ID NO: 11]

| | | |
|---|---|---|
| FP (b50-3f): | ctatcactgctgggtgcaaa | (SEQ ID NO: 38) |
| RP (b50-3r): | catcccatcaatcacagtgc | (SEQ ID NO: 39) |

Human glyceraldehyde-3-phosphate dehydrogenase (GAPDH) gene and human β-actin gene were used as internal standards. Real-time PCR primers for these genes had the following sequences:

| | | |
|---|---|---|
| GAPDH (FP): | cggctactagcggttttacg | (SEQ ID NO: 40) |
| GAPDH (RP): | aagaagatgcggctgactgt | (SEQ ID NO: 41) |
| B-actin (FP): | aaaactggaacggtgaaggtg | (SEQ ID NO: 42) |
| B-actin (RP): | tgtgtggacttgggagagga | (SEQ ID NO: 43) |

All of the primers used in the analysis were synthetic DNA primers (Nisshinbo Industries). The temperature cycle of the real-time PCR was as follows: step 1=50° C., 2 min; step 2=95° C., 10 min; step 3=95° C., 15 sec; step 4=60° C., 1 min; and step 5=40 cycles of step 3 and step 4.

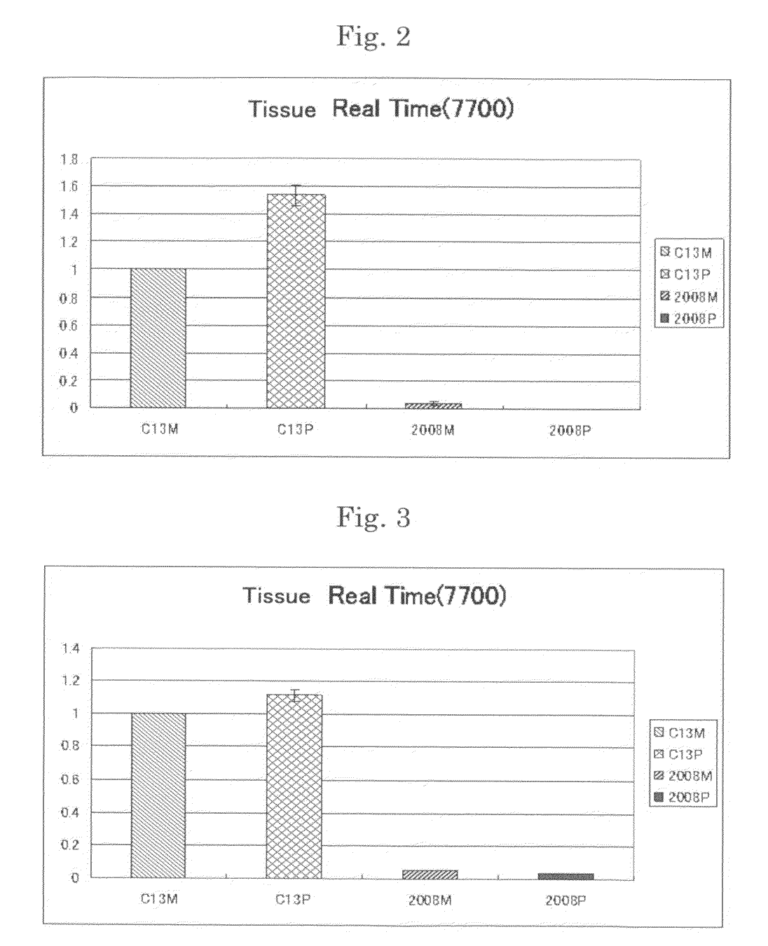

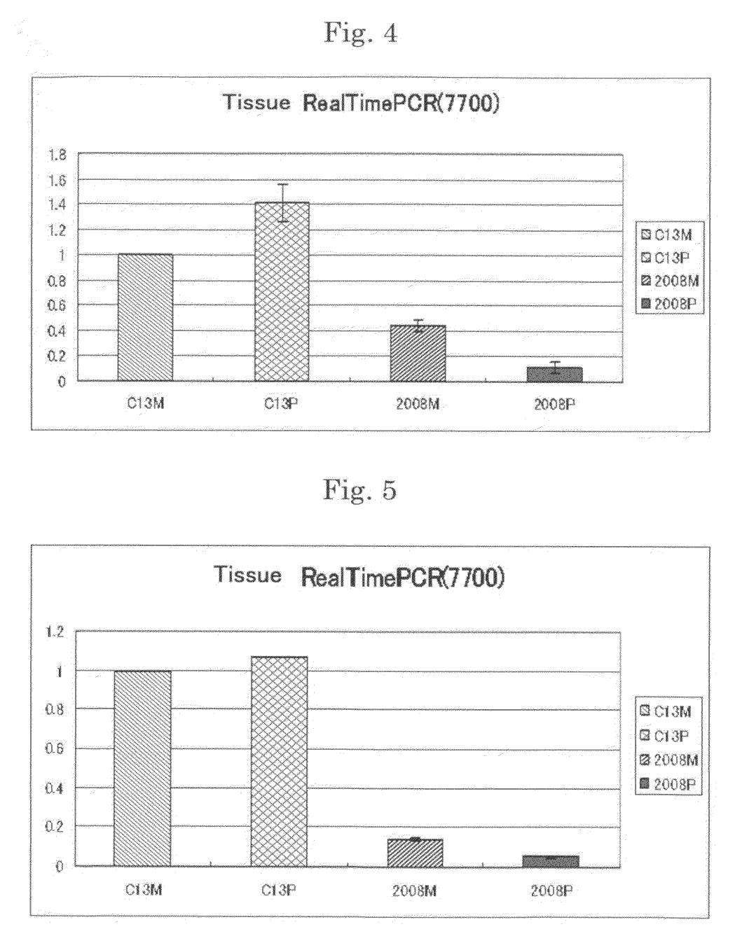

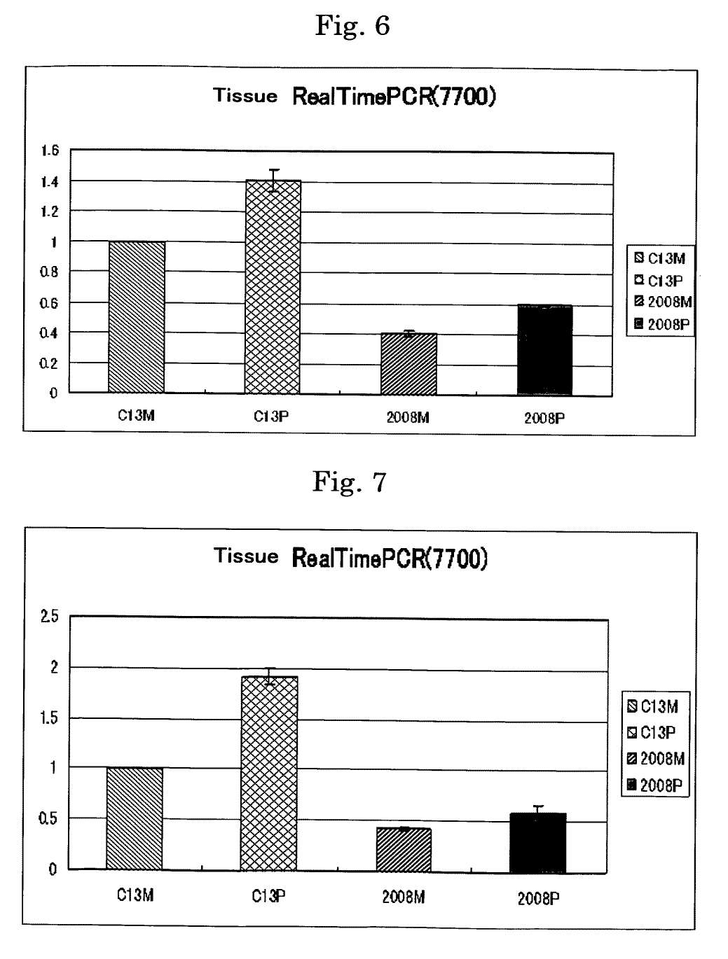

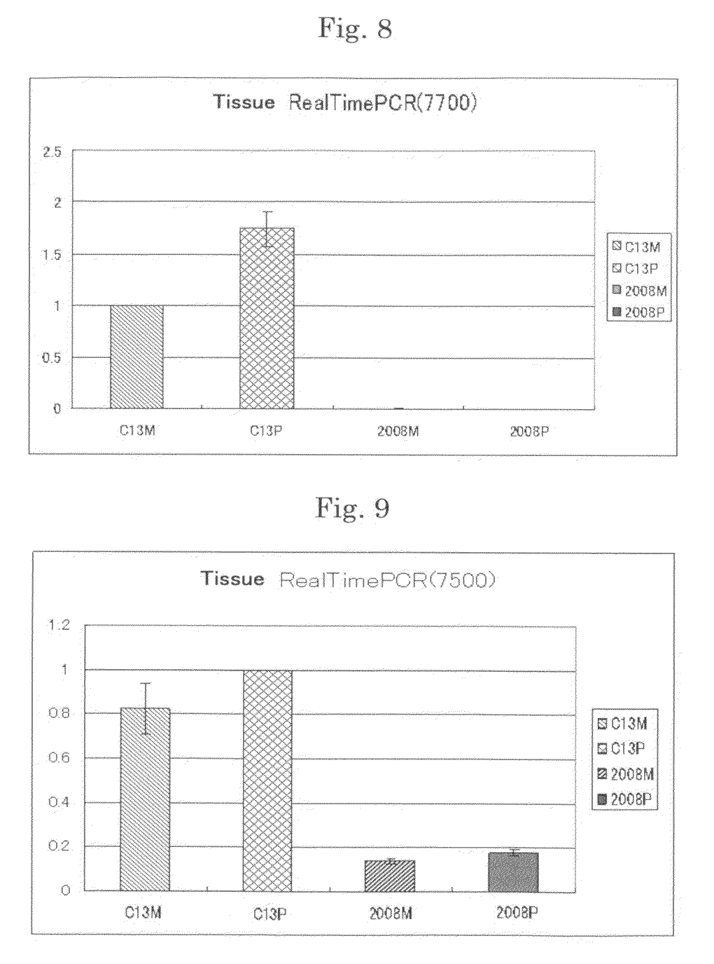

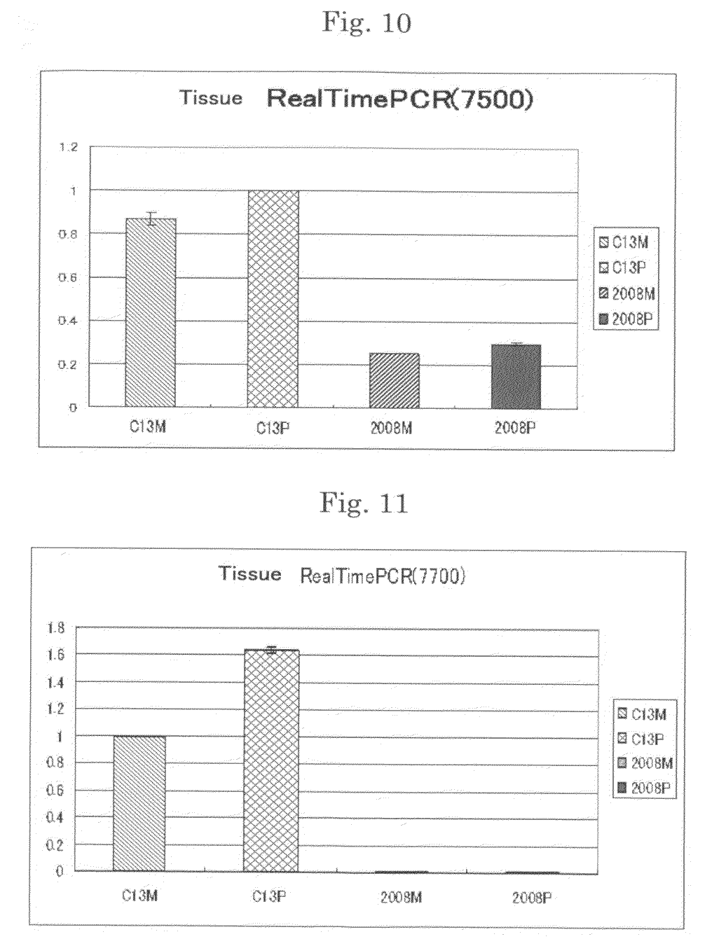

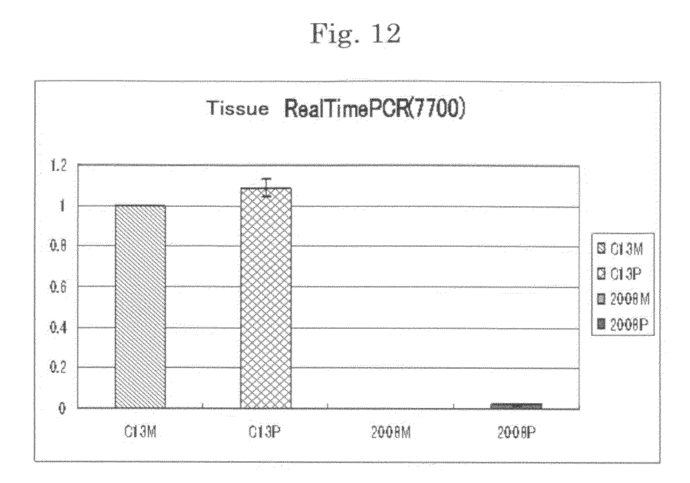

The results of real-time PCR of the genes obtained above (cDNA fragments of SEQ ID NOs. 1 to 11) are shown in FIGS. 2 to 12. In the graphs shown, C13M and C13P indicate C13 (−) and C13 (+), respectively, and 2008M and 2008P indicate 2008 (−) and 2008 (+), respectively. These results indicate that all of the genes shown (cDNA fragments of SEQ ID NOs.1 to 11) are expressed specifically in the cisplatin-resistant ovarian tumor.

The cisplatin-resistance marker of the present invention also includes polynucleotides having a base sequence that hybridizes with a polynucleotide having any one of the base sequences of SEQ ID NOs.1 to 11 under stringent conditions. The term “stringent conditions” as used herein includes, for example, at 37° C. to 80° C., 0.05 to 5×SSC and 0.1 to 10% SDS, and preferably at 50° C. to 72° C., 0.1 to 2×SSC and 0.2 to 5% SDS.

The cisplatin-resistance marker of the present invention also includes polynucleotides having a base sequence complementary to all or part of a polynucleotide having any one of the base sequences of SEQ ID NOs. 1 to 11. The term “complementary” is used herein to describe not only exactly matching base sequences, but also those with at least 70% or higher, preferably at least 80% or higher, more preferably at least 90% or higher, and still more preferably at least 95% or higher sequence similarity.

### (Cisplatin-Resistance Marker)

The cisplatin marker of the present disclosure may be used as a tool to determine whether a given ovarian tumor is resistant to cisplatin. The marker may be a polynucleotide having any of the base sequences of SEQ ID NOs. 1 to 11 or a polynucleotide having a complementary sequence to any of the base sequences of SEQ ID NOs. 1 to 11. The marker may be a single strand or a double strand of these polynucleotides. The entire set of the polynucleotides of SEQ ID NOs. 1 to 11 may be used as a marker set for the cisplatin-resistant ovarian tumor. Alternatively, any number of the polynucleotides of SEQ ID NOs. 1 to 11 may be used in combination as a cisplatin resistance marker set.

According to the present invention, the diagnosis of cisplatin resistance is made by determining the presence or absence, or the level of the expression of at least one of the genes of SEQ ID NOs. 1 to 11 in the living tissue or cultured cells of a subject. The marker of the present invention specifically recognizes RNA expressed by the above-described genes or polynucleotides derived from such RNA and can therefore be used as a primer to amplify them or as a probe to specifically recognize and detect them.

When used as a primer, the polynucleotide of the present invention has a base sequence with a length of typically 15 bp to 100 bp, preferably 15 bp to 50 bp, and more preferably 15 bp to 35 bp. When the polynucleotide of the present invention is used as a probe, such a probe includes at least part or all of the polynucleotide and has a length of from at least 15 bp to the full length of the polynucleotide. When used as a primer, the polynucleotide of the present invention may be fluorescence-labeled, radiolabeled or labeled with any other proper label. These labels can be introduced by any known technique.

The marker of the present invention may be used as a primer or a probe in northern blotting, in situ hybridization, RT-PCR or any other known technique designed to specifically detect genes of interest. In these techniques, the primer or the probe may be used to determine the presence or absence, or the level of the expression of the genes of the present invention. The present invention may be applied to total RNA obtained by known techniques from the living tissue or cultured cells of a subject, or polynucleotides prepared from such RNA.

The marker of the present invention may be used in conjunction with a microarray (DNA chip) to determine the resistance to cisplatin. In such a case, the marker (polynucleotide) of the present invention is used as a probe (25 bp in length, for example) that is immobilized on a microarray and hybridizes with labeled DNA or RNA prepared from RNA from the living tissue or the like. The complex formed by hybridization of the probe (i.e., the marker of the present invention) with the labeled DNA or RNA can be detected due to the presence of the label in the labeled DNA or RNA. In this manner, the presence or absence, or the level of the expression of the genes related to the cisplatin resistance can be determined in the living tissue or the like.

### (Method for the Detection (Diagnosis) of Cisplatin-Resistant Ovarian Tumor)

The detection method of the present invention detects the presence or absence, or the level of the expression of the genes related to cisplatin resistance in samples collected, by known techniques, from the living tissue or cultured cells of a subject with an ovarian tumor. The diagnosis of resistance (sensitivity) of the ovarian tumor (cells) to cisplatin can then be made based on the detected results. The sample for use in the detection method may be either total RNA or polynucleotides prepared from such RNA.

Specifically, the detection (diagnosis) method of the present invention involves the following steps: (1) hybridizing RNA prepared from a biological sample from a subject with an ovarian tumor, or a complementary polynucleotide transcribed from such RNA, with the cisplatin-resistant ovarian tumor marker of the present invention; (2) quantifying the RNA of the biological sample or the complementary polynucleotide (transcribed from the RNA) that has hybridized with the marker, using the marker as an index; and (3) based on the results of the quantification, determining whether the ovarian tumor is resistant to cisplatin.

The marker used in Step (2) may be a primer or probe designed based on at least one of the polynucleotides of SEQ ID NOs. 1 to 11 and/or a polynucleotide complementary to any of the polynucleotides of SEQ ID NOs. 1 to 11.

For example, the marker of the present invention may be used as a primer in RT-PCR for detecting the presence or absence, or the level of the expression of the genes of the present invention that are related to cisplatin resistance.

### INDUSTRIAL APPLICABILITY

The cisplatin-resistance marker of the present invention makes it possible to determine whether a given human ovarian cancer is resistant to cisplatin.

What is claimed is:

1. A cisplatin-resistance marker for ovarian tumor, comprising:
a polynucleotide having any one of the base sequences of SEQ ID NOs. 1 to 11.

2. A cisplatin-resistance marker for ovarian tumor, comprising:
a polynucleotide having a base sequence that hybridizes with a polynucleotide having any one of the base sequences of SEQ ID NOs. 1 to 11 under stringent conditions.

3. A cisplatin-resistance marker for ovarian tumor, comprising:
a polynucleotide having a base sequence complementary to all or part of a polynucleotide having any one of the base sequences of SEQ ID NOs. 1 to 11.

4. The cisplatin-resistance marker for ovarian tumor according to claim 1, wherein the marker is used as a probe to detect a cisplatin-resistant ovarian tumor.

5. The cisplatin-resistance marker for ovarian tumor according to claim 1, wherein the marker is used as a primer to detect a cisplatin-resistant ovarian tumor.

6. A microarray for detecting a cisplatin-resistant ovarian tumor, comprising:
a cisplatin-resistance marker for ovarian tumor, which is used as a primer to detect a cisplatin-resistant ovarian tumor.

7. A method for detecting a cisplatin-resistant ovarian tumor, comprising:
(1) hybridizing RNA prepared from a biological sample from a subject, or a complementary polynucleotide transcribed from the RNA, with the cisplatin-resistance marker according to claim 4;
(2) quantifying the RNA from the biological sample, or the complementary polynucleotide transcribed from the RNA, that has hybridized with the cisplatin-resistance marker, using the cisplatin-resistance marker as an index; and
(3) determining whether the biological sample is a cisplatin-resistant ovarian tumor based on the results of the quantification.


SEQUENCE LISTING (43 sequences)

SEQ ID NO: 1 - DNA, 19 bp, human
  Inventor Moriaki, Kusakabe
ccggattggagtgtcttaa

SEQ ID NO: 2 - DNA, 30 bp, human
ccggtgactggacatatcacctctacttaa

SEQ ID NO: 3 - DNA, 55 bp, human
ccggtactgggtgttcctgctcatcagacccctaaccttgcaagaccatcattaa

SEQ ID NO: 4 - DNA, 55 bp, human
ccggccgatgggcttagctgtagcttactgaagtcgccaagcaggagagatttaa

SEQ ID NO: 5 - DNA, 66 bp, human
ccggagctgcaatctagtcctgcctcctgtccgccatgatcccgatcactggttgcataaggttaa

SEQ ID NO: 6 - DNA, 70 bp, human
ccggcttgctggcttgcccttcacattgcagaattgccagcctgcacaattgtgtgggccaagttcttaa

SEQ ID NO: 7 - DNA, 117 bp, human
ccgggcgggaaagtgggagtggcaggaatgaaacaggaaaaggagtctggactcaatgcatgctagggattttggacatcattgcgtggacaacttgaaatcatggaaggtttttaa

SEQ ID NO: 8 - DNA, 138 bp, human
ccggtggaggagagcgccttggaacgccgacctgaaaccatctctgagcccaagacctaagaagctgagaacagaggtctgcagagagacaatgaagcagagggaagttgaagtgtaggattgagagaaagcccttaa

SEQ ID NO: 9 - DNA, 263 bp, human
ccggccatgtctgaccccaatcggccaccatgtcgcaagaatgatgatgacagcagctttgtagatggaaccaccaacggtggtgcttggtacagcgtacctggagggatgcaagacttcaattaccttagcagcaactgttttgagatcaccgtggagcttagctgtgagaagttcccacctgaagagactctgaagacctactgggaggataacaaaaactccctcattagctaccttgagcagatacaccgaggagttaa

SEQ ID NO: 10 - DNA, 268 bp, human
ccggtggttcccagcgagggggttggagaagaaggtccctcaaccaacagtaggctcgagttcaggcacaccctgtcctcaggagcctggctcccctacccaacccagccctatcactgatgggccatgtgctccctcactccctctttctgtgcctcagtttcctcttctgctgaggagactctaatccctgccccatggtcttcctcataggcaatgcagttgtgaggctcaaatgagatagattatggtgataaagaggttttaa

SEQ ID NO: 11 - DNA, 330 bp, human
ccgggccatttctgagagtggcgtggccctcacttctgttctggtgaagaaaggtgatgtcaagcccttggctgagcaaattgctatcactgctgggtgcaaaaccaccacctctgctgtcatggttcactgcctgcgacagaagacggaagaggagctcttggagacgacattgaaaatgaaattcttatctctggacttacagggagaccccagagagagtcaaccccttctgggcactgtgattgatgggatgctgctgctgaaaacacctgaagagcttcaagctgaaaggaatttccacactgtcccctacatggtcggaattaa

SEQ ID NO: 12 - DNA, 28 bp, Artificial Sequence
  Description of Artificial Sequence  Adapter
aatggctacacgaactcggttcatgaca

SEQ ID NO: 13 - DNA, 30 bp, Artificial Sequence
  Description of Artificial Sequence  Adapter
cgtgtcatgaaccgagttcgtgtagccatt

SEQ ID NO: 14 - DNA, 28 bp, Artificial Sequence
  Description of Artificial Sequence  Adapter
aagtatcgtcacgaggcgtcctactgcg

SEQ ID NO: 15 - DNA, 30 bp, Artificial Sequence
  Description of Artificial Sequence  Adapter
tacgcagtaggacgcctcgtgacgatactt

SEQ ID NO: 16 - DNA, 20 bp, Artificial Sequence
  Description of Artificial Sequence  Primer
actcggttcatgacacggnn

SEQ ID NO: 17 - DNA, 20 bp, Artificial Sequence
  Description of Artificial Sequence  Primer
aggcgtcctactgcgtaann

SEQ ID NO: 18 - DNA, 20 bp, Artificial Sequence
  Description of Artificial Sequence  Primer
cggattggagtgtcttaacg

SEQ ID NO: 19 - DNA, 20 bp, Artificial Sequence
  Description of Artificial Sequence  Primer
cagccaccatagcaggaaca

SEQ ID NO: 20 - DNA, 20 bp, Artificial Sequence
  Description of Artificial Sequence  Primer
ttccagttgactgcagagga

SEQ ID NO: 21 - DNA, 20 bp, Artificial Sequence
  Description of Artificial Sequence  Primer
tcgctaaacaggacggattt

SEQ ID NO: 22 - DNA, 20 bp, Artificial Sequence
  Description of Artificial Sequence  Primer
tgtagatggcaggttgatgg

SEQ ID NO: 23 - DNA, 20 bp, Artificial Sequence
  Description of Artificial Sequence  Primer
ggttaggggtctgatgagca

SEQ ID NO: 24 - DNA, 20 bp, Artificial Sequence
  Description of Artificial Sequence  Primer
cttactgaagtcgccaagca

SEQ ID NO: 25 - DNA, 20 bp, Artificial Sequence
  Description of Artificial Sequence  Primer
tgtgcgatatttgacccttg

SEQ ID NO: 26 - DNA, 20 bp, Artificial Sequence
  Description of Artificial Sequence  Primer
cggagctgcaatctagtcct

SEQ ID NO: 27 - DNA, 20 bp, Artificial Sequence
  Description of Artificial Sequence  Primer
attcgccacagcttttcaat

SEQ ID NO: 28 - DNA, 20 bp, Artificial Sequence
  Description of Artificial Sequence  Primer
atgctgctgtgaaagtgtgc

SEQ ID NO: 29 - DNA, 20 bp, Artificial Sequence
  Description of Artificial Sequence  Primer
caggctgggtttcttctctg

SEQ ID NO: 30 - DNA, 20 bp, Artificial Sequence
  Description of Artificial Sequence  Primer
ggcaggaatgaaacaggaaa

SEQ ID NO: 31 - DNA, 20 bp, Artificial Sequence
  Description of Artificial Sequence  Primer
gatttcgttgaccccatcac

SEQ ID NO: 32 - DNA, 20 bp, Artificial Sequence
  Description of Artificial Sequence  Primer
ccgacctgaaaccatctctg

SEQ ID NO: 33 - DNA, 20 bp, Artificial Sequence
  Description of Artificial Sequence  Primer
aagggctttctctcaatcct

SEQ ID NO: 34 - DNA, 20 bp, Artificial Sequence
  Description of Artificial Sequence  Primer
gctcctggtcatcgagctgt

SEQ ID NO: 35 - DNA, 20 bp, Artificial Sequence
  Description of Artificial Sequence  Primer
ttgtctcgttccccttctgg

SEQ ID NO: 36 - DNA, 20 bp, Artificial Sequence
  Description of Artificial Sequence  Primer
tggagaagaaggtccctcaa

SEQ ID NO: 37 - DNA, 20 bp, Artificial Sequence
  Description of Artificial Sequence  Primer
gggcagggattagagtctcc

SEQ ID NO: 38 - DNA, 20 bp, Artificial Sequence
  Description of Artificial Sequence  Primer
ctatcactgctgggtgcaaa

SEQ ID NO: 39 - DNA, 20 bp, Artificial Sequence
  Description of Artificial Sequence  Primer
catcccatcaatcacagtgc

SEQ ID NO: 40 - DNA, 20 bp, Artificial Sequence
  Description of Artificial Sequence  Primer
cggctactagcggttttacg

SEQ ID NO: 41 - DNA, 20 bp, Artificial Sequence
  Description of Artificial Sequence  Primer
aagaagatgcggctgactgt

SEQ ID NO: 42 - DNA, 21 bp, Artificial Sequence
  Description of Artificial Sequence  Primer
aaaactggaacggtgaaggtg

SEQ ID NO: 43 - DNA, 20 bp, Artificial Sequence
  Description of Artificial Sequence  Primer
tgtgtggacttgggagagga